(19) United States
(12) Patent Application Publication
(10) Pub. No.: US 2021/0038911 A1
(43) Pub. Date: Feb. 11, 2021

(54) Method and Apparatus of Providing Wavefront Color Therapies and Applications

(71) Applicants: Dara C. Medes, Woodbridge, CT (US); Heather Lyn Medes, Woodbridge, CT (US)

(72) Inventors: Dara C. Medes, Woodbridge, CT (US); Heather Lyn Medes, Woodbridge, CT (US)

(21) Appl. No.: 16/532,355

(22) Filed: Aug. 5, 2019

(51) Int. Cl. A61N 5/06; A61M 21/00

(52) U.S. Cl. CPC A61N 5/0618; A61M 21/00; A61N 2005/0648; A61M 2021/005; A61N 2005/0663; A61N 5/0616

(57) ABSTRACT

A method and apparatus of providing wave-front color therapy using a computer or handheld smart device to deliver a specific nanometer wavelength of light to affect a wide variety of imbalances that interfere with function and performance. Specific wavelengths of light are delivered through handheld devices such as smartphones, tablets, smartwatches, and Virtual Reality headgear or by computerized screen display. The exact nanometer specifications and hue-saturation will be determined by the practitioner, specialist or educator using this device. These benefit individuals with learning disabilities, circadian rhythm disruption and sleep disturbances, jet lag, Shift Work Sleep Disorder, and computer screen blue light exposure. Also Seasonal Affective Disorder, skin disorders, hematological and bilirubin levels, tinnitus, and neurological disease and disparity, to name several. This disclosure claims analog and digital relations of light as it relates to both the spatial and temporal relationship of light.

### CROSS-REFERENCE TO RELATED APPLICATIONS

This application is a continuation-in-part of U.S. patent application Ser. No. 10/713,912 filed May 2, 2003 now U.S. Pat. No. 7,253,824.

### STATEMENT REGARDING FEDERALLY SPONSORED RESEARCH OR DEVELOPMENT

N/A

### THE NAMES OF THE PARTIES TO A JOINT RESEARCH AGREEMENT

N/A

### INCORPORATION-BY-REFERENCE OF MATERIAL SUBMITTED ON A COMPACT DISC OR AS A TEXT FILE VIA THE OFFICE ELECTRONIC FILING SYSTEM

N/A

### STATEMENT REGARDING PRIOR DISCLOSURES BY THE INVENTOR OR A JOINT INVENTOR

N/A

### BACKGROUND OF THE INVENTION

### 1. Field of Invention

The invention relates to human/computer interfaces on desktop computers and portable devices. The current invention has diagnostic use and produces color therapy programs for patient, student or consumer output. The apparatus produces a color therapy wavelength shift display that can rapidly pinpoint a useful color therapy sequence for a variety of conditions and issues. The optimal color sequence can be recorded and distributed for individual use on their personal desktop, laptop or handheld device. This method and apparatus provides wave-front color therapy using a computer or portable handheld smart device to deliver a variety of specific different nanometer wavelength of light sequences for a precise treatment or effect. These specific color therapy sequences can be displayed simultaneously, in sequence or individually. Another display option is as a partially transparent overlay concurrent with other beneficial programs for optimal effect, including 3D displays and patient or other user interactive usage. This is useful for various health, wellness, academic, and lifestyle difficulties that may benefit from the delivery of different wavelengths of light through a handheld smart device such as a smart phone or tablet, or via a computer monitor or comparable computerized screen display.

The use of smart or intelligent handheld devices to display pre-recorded applications of color therapy sequences is disclosed in U.S. Pat. No. 7,253,824 (“the ‘824’ patent”).

The ‘824’ patent cites the use of said internet-enabled, smart or intelligent portable handheld devices as a potential color therapy program display option for end-user use. Claim 9 described using handheld devices to display the color therapy sequences created for the user. These sequences were originally only diagnosed, recorded and stored on a desktop computer. The sequences were then transferred to the user via the internet or on a data storage medium for use on a computer or handheld device.

Portable devices include PDAs, intelligent or smart tablet PCs, smartphones and other similar telecommunication systems as well as internet equipped smart devices with screen display worn on the person. Some examples include internet-enabled Smart Watches and pendants and other similar wireless telecommunication systems. Such devices can provide portable software systems intended for rehabilitation, relaxation, wellness, reading enhancement, and other documented uses of color therapy. These modern handheld devices can greatly expand the functionality and display options of the claim of the ‘824’ patent because of the following features:
1. The Operating Systems and CPU capabilities of modern handheld devices are often as robust as laptop and desktop systems. Modern handheld devices can execute multiple programs concurrently without system comprise or lag. They can support extended program features. Some examples include uninterrupted streaming animation, movies, file editing, processing, and video conference.
2. Smartphones, smart watches and tablets can connect, cast or stream to a variety of other display screens and devices. This can include larger screens or monitors, televisions and Virtual Reality (VR) headgear capable of displaying 3D motion output. This makes said handheld devices a useful means of displaying the extended capabilities of the ‘824’ patent invention.
3. Modern handheld devices use both internal and cloud storage to download, store and send larger program files. These files can take the form of downloadable mobile applications, or “apps”. This includes programs such as the stored color therapy sequences of the ‘824’ patent claim.

Technology has advanced these devices to include input and output mechanisms capable of expanding the claimed invention of the ‘824’ patent and even layering multiple programs together to increase productivity, rehabilitation, therapeutic benefit and overall functionality. These include smart watches, pendants, and virtual reality headsets capable of displaying or casting 3D motion output. With small program additions, a portable device can be paired with virtual reality headset glasses to create a simulated display for use of the previously claimed invention and extended 3D functionality.

The implication of these handheld device capabilities to the 824 patent claims includes the following factors:

First, with minor expanded feature developments to the previously claimed invention of the ‘824’ patent, handheld devices can be used as both a diagnostic or end-user color therapy tool that combines with alternate display means.

Second, expanded device display power now allows the previously claimed invention to display multiple concurrent instances of semi-transparent color therapy overlays in conjunction with other programs or overlays of the same or other authors.

Third, display mechanisms involve download, casting, streaming and connecting to external display units.

Fourth, handheld devices including tablets now uniformly support touchscreen capability and audio command support, compensating for very young or older user's mobility issues.

Fifth, handheld devices paired with external display screens can display or cast a 3D display of color therapy combined with other animations or programs of the same authors (Dara C. Medes, Heather Lyn Medes). These can be for rehabilitation, educational, wellness, lifestyle or entertainment purposes.

These and other advents in handheld technologies offer a simplified approach to the existing protocol, and expand the potential uses of the previously claimed invention beyond the originally claimed configurations.

These extensions can be in the form of adding a few expanded features to the previously claimed invention, and to use said invention output displayed concurrently as a transparent overlay along with other programs of the same or other authors.

Pairing the color shift therapy apparatus with modern smartphone or tablet features allows for expanded color therapy options and capabilities. The optimized color therapy program can be combined with partially transparent programs overlaying other applications or programs. These applications can include educational, training or treatment programs, text content and data, image or animation, 3D animation and renderings, film, film effects, motion displays or streaming data, combined with audio component and/or audio response feedback.

The ‘824’ Patent also describes the diagnostic use of the original invention as confined to in-office use. This was in part necessitated by the non-portable nature of the claimed hardware configuration. A simpler means of diagnostic use is by employing a handheld device, such as an internet-enabled tablet PC. User input was formerly only obtainable by keyboard, mouse or joystick. A tablet or other modern handheld device uses an interactive touchscreen. Touchscreens offer intuitive and exact selection capabilities. This is diagnostically useful. It is also easier for end-users with dexterity issues, notably very young or older users.

Modern handheld devices simplify the diagnostic process described in the ‘824’ patent. Diagnostic use is no longer limited to in office desktop configurations. Tablets and other handheld devices provide a portable diagnostic means via a tablet and other handheld devices, including various incarnation of laptop/tablet hybrids where the screen tablet detaches for portable screen use.

Advances in touchpad technology offer interactivity unavailable with the previously claimed hardware configuration. For example, the ability to rapidly adjust color sequences and transparencies with precision or document user reaction time to on-screen events is useful for diagnostic or training purposes.

It also expands the potential uses of the originally claimed invention to include any application where color therapy may enhance the productivity, health benefits, or enjoyment of another program.

For the reasons cited above, the invention of the ‘824’ patent claim can thereby also be used in a more simplified and portable manner. This application is useful where color therapy or color therapy paired with inventions of the same (Dara C. Medes, Heather Lyn Medes) or other authors would benefit from a simplified approach of one unit for screen display, CPU, and program data storage with internet access to share data and color sequence programs. While the original desktop diagnostic configuration may still be used, a portable color therapy diagnostic tool can provide extended benefits of portability and ease of interactivity, even amongst those with limited dexterity. This allows for not just in-office use, but anywhere color therapy overlaid with other programs would be useful, including educational settings, reading applications, computerized ocular precision training for sports such as golf, and the like.

This expands the scope of programs originally designed for rehabilitation and learning disability enablement to include other uses which have proven beneficial to rehabilitation, learning disabilities, and the general population. The ability to provide an affordable, portable, comfortable and convenient means of color therapy has proven beneficial in a number of settings and applications. Some of these applications include pre-designed color therapy sequences with documented benefits, custom color therapy sequence creation, diagnosis of optimal color overlays or precision color to best alleviate symptoms associated with reading difficulties and custom color therapy sequence partial transparent overlay on digital textbook and learning materials.

Technological advances in portable devices allow for the execution and display of multiple concurrent software applications. This allows for the use of the original software application displayed over other useful applications of the same or of other authors. A small addition of the ability to adjust the color therapy display opacity levels can be made in order to display the color therapy program as a partial transparency. This allows the previously claimed invention, adjusted by said additions, to serve as a colored overlay to digital textbooks and reading material, as well as to function as a diagnostic test for optimal color overlay assessment during functional reading testing.

### 2. Prior Art

Prior art is divided into four primary categories: diagnostic tools, educational/learning and reading enhancement, rehabilitation treatment methods, and previously designed light therapy treatment methods.

The first area of prior art is the manner of diagnostic tools used in the development of wave-front color therapy for therapeutic purposes. To date, optimal color therapy selection has been a manual, often tedious process not capable of pinpointing the precise nanometer of a colors wavelength best suited for the end user's use.

The second area of prior art is the manner of rehabilitation treatment methods for neurological impairments and learning disabilities such as Attention Deficit Disorder, and ADHD.

There is a host of computer based, non-portable, dumb terminal rehabilitation systems used within the structure of cognitive and learning disability rehabilitations. They are geared at re-training the injured, disadvantaged, diseased or stressed neurological processes.

There are basic shortcomings to the conventional approach of office-based rehabilitation, retraining, learning and therapy sessions. These machines are only available to the educational or rehabilitation facility due to cost and size and are therefore unavailable for private patient, student or other end-user consumer use. This limits the amount of time a patient, client or other end-user can spend using these color therapy tools.

This time can be recaptured with a portable rehabilitation device to make best use of time.

A patient or other end-user can use this device to maximize their rehabilitation, reducing rehabilitation expense while making best use of the window of maximum rehabilitative progress.

Third, a fundamental problem in the conventional approach is that is does not fully take into account the need of the learning-impaired student. Learning disabilities include but are not limited to neurologically-based processing problems.

A learning-impaired student is paired with a learning specialist during school hours, which either robs time from their education or uses their break periods, leaving an already overworked student without a break during the day. The second approach is to team a student with a learning specialist after school, taking time away from homework and putting a student further behind in their work. A learning-impaired student's time is divided and at a premium.

Any adaptive technology devices that a mainstreamed student may be offered might not be available in all schools, and a student may often be embarrassed to use them in front of others students who may perceive a learning disability as a lack of intelligence. Fear of such a perception may render a student reluctant or too embarrassed to use the adaptive tech tools designed to help them.

Colored overlays are used for reading enhancement and accommodation of various learning disabilities. To date, these colored overlays are individual film sheets placed atop print materials, such as classroom handouts and textbooks. They do not accommodate digital eReaders, phones, tablets, or other digitally based textbooks and eBooks. They also do not accommodate interactive learning materials. While a colored overlay could theoretically be placed over a computer screen, portable screen or tablet device, there are limitations in attempting to adapt this traditional approach to modern text displays.
1) Tape or static electricity is necessary to place the overlay on the screen.
2) The overlays are designed for books or papers, and are not screen width. This leaves gaps or requires multiple overlays with a visible seam.
3) The color does not remain true. There is a lack of control of computer or tablet color “white light”, colored or textured background cannot be accommodated for, resulting in an unwanted projected color mix.
4) Brightness and contrast must be adjusted for clarity.

The present invention offers a computer-based overlay that can calibrate for proper color overlay display without distortion. There are no gaps in sizing or need for adhering the overlay to computer hardware. The computer display can also provide a gentle color shift to encourage a slower breathing pace for relaxed reading.

Another use of Color Overlays or color therapy is to negate the impact of blue light from screen displays. Blue light screens have been associated with circadian rhythm disturbance and disrupted sleep patterns. This has become increasingly problematic as smartphone and computer device use increases. The negative visual impact of blue light can be largely negated with a semi-transparent color overlay or gradient color overlay of the current invention in a specific wavelength shift of ˜590-625 nm running concurrently atop other programs and applications.

Color or Light Therapy is used for a number of purposes. Some uses are prescribed with an office or educational setting. Others uses are available outside of a medical setting and mass marketed for specific problems. An example would be light therapy lamps or boxes for at-home use geared towards Seasonal Affective Disorder (SAD). Color therapy has been found beneficial for health, wellness, lifestyle, and educational applications. Users include patients, the learning disabled and the general population who may benefit from color therapy to improve function, relaxation, sleep, or enjoyment. Color Therapy has long been used medically for uses such as Seasonal Affective Disorder (SAD), dermatological purposes, cosmetic enhancement, as well as for Syntonic Optometry. The latter has been used for the past 80 years for treatment of several optometric disorders. Color Therapy has been used in the diagnosis and treatment of brain injury, cerebro-vascular accident and other neurological disorders and motor-related neurological conditions. Specific wavelengths of light have been found beneficial for a variety of issues. This includes color therapy sequences have proven beneficial to those suffering from circadian rhythm disturbance such as jet lag and Shift Work Sleep Disorder. Studies show a deep red light of 650 nm to 900 nm may stimulate mitochondrial repair. A promising 2019 study shows green light at a wavelength of 500 nm effective in cancer pain management. [1]

There are, however, several failings of the treatments and therapies developed to date:
1. White light machines. Many light machines emit full spectrum white light, not specific and finite wavelengths. There are multiple benefits to being able to isolate a finite wavelength, as in the case of this claimed computer program:
   a. The white light machines available today, by their very nature, emit all wavelengths in the visible spectrum. For as therapeutic as certain wavelengths of color can be to a patient, another wavelength could be harmful or uncomfortable, and there is no way to omit the uncomfortable or harmful wave lengths from a white-light machine and only use the helpful ones for therapy.
   b. Many suffer from photophobia or sensitivity to light and glare. While some white light machines have a dimmer, this may not reduce brightness and glare enough for the end-user or patient and may cause discomfort, and would not be therapeutic.
   c. Since all colors are emitted from a white light machine, it is impossible to determine what wave lengths could be most helpful to the patient. In contrast, this computer program can isolate the exact wavelength of color that is beneficial to the patient.
   d. White light machines often require extended periods of time per day to receive therapeutic benefit. By this program isolating to the most therapeutic range of wavelengths, the patient will receive the most precise diagnosis and the best therapy for their specific disorder in the shortest amount of time. This is essential as there is a limited window of time after neurological injury or onset of a neurological illness that a patient has to capture the majority of recovery they will make—thus, time is of the essence.
2. Methods of color therapy developed to date that isolate certain color spectrums are generally unable to provide the diagnostic benefits of the computer program claimed herein due to their inability to produce the scope of colors necessary. In addition, they also lack certain elements of the ideal color manipulation.
   One such example is the use of lasers and radiation of certain colors on the eye, with the obvious side effects associated with lasers and radiation. Other methods of light therapy involve physically dangerous illumination apparatuses such as gas or flame, which are dangerous and prohibit unattended or at home use due to their very nature.
   Discomfort of use experienced by excess heat output is another problem of current light therapy devices. Several at-home use light therapy devices used for Sleep Disorders, Seasonal Depression and similar uses generate excessive heat, causing user discomfort. This discourages the necessary consistent use. None of these factors are an issue with the current claimed invention.

### BRIEF SUMMARY OF THE INVENTION

It is therefore an object of the current invention to use current and future computer and telecommunication hand held and mobile devices as a method of transportable rehabilitation, light therapy treatments, and a diagnostic device. Using said devices is especially useful as patients and other end users likely already own them, requiring limited additional investment to benefit from color therapy programs. Such a device is far more cost effective than any other rehabilitation device currently available.

Educational or office-based color therapy can be adapted for handheld device use. Such software can be purchased or downloaded to the handheld device via the Internet or from the rehabilitation office or educational setting. This allows the rehabilitation office or educational setting to provide consistent rehabilitation or assistance when a patient, student or other user is unable to attend. This would require a software suite available to the office or educational facility, as well as a website for download of software to the handheld device.

It is still a further object of the present invention that it provides a computer display for users that is convenient, lightweight, low-cost, minimally power hungry, and capable of portable operation without degraded performance.

In addition to the objects above, and in all handheld or otherwise portable devices useful in the present invention, less portable means of display such as laptop computers, desktop computers, televisions, or any other telecommunication or display device are also useful in the present invention. These include screen displays capable of receiving casted transmissions.

A color light therapy computer method and apparatus has been produced with the capability to display a full range of wavelengths systematically delineated of the visual spectrum. Using said program claimed herein as a foundation application, with various modifications, the preciseness of the wave length production and display thereof allows for a diagnostic process and a host of rehabilitative and treatment applications to be produced from the same fundamental program.

### BRIEF DESCRIPTION OF THE SEVERAL VIEWS OF THE DRAWINGS

The above-mentioned features and objects of the present invention will become more apparent with reference to the following description taken in conjunction with the accompanying drawings wherein like reference numerals denote like elements and in which:

FIG. 1 is a table illustrating the visible colors spectrum shifting combinations used in a computer program for a computer capable of at least 256,000,000 colors:

FIG. 2 is a block diagram for an apparatus in accordance with the teachings of the original invention;

FIG. 3 is a flow diagram illustrating the method of present invention; and

FIG. 4 is a flow diagram illustrating the shifting of the combination of the visible color spectrum in a computer program of the present invention.

FIG. 5 is a is a block diagram for an apparatus in accordance with the teachings of the present invention; demonstrating color therapy use on a desktop device, as well as modern handheld telecommunication devices such as smartphones, smart watches and tablets, where casting to portable screen displays or for use with Virtual Reality (VR) headset gear or glasses can create a simulated or 3D display.

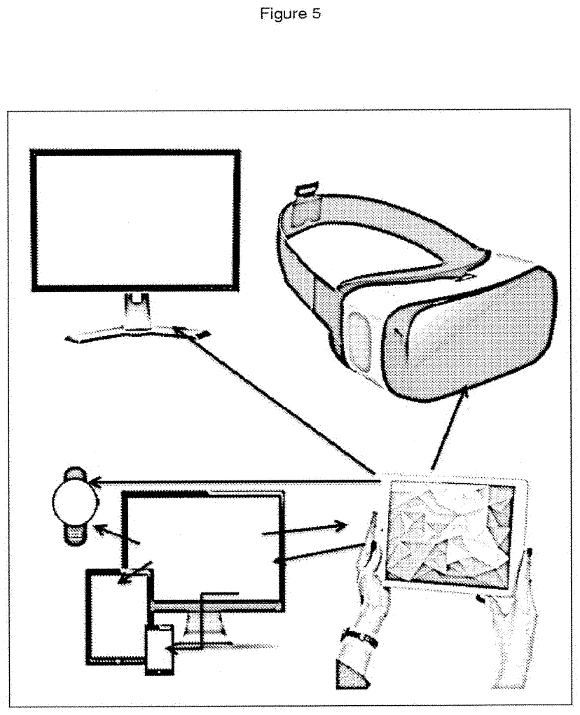

### DETAILED DESCRIPTION OF THE INVENTION

Although the visual system has been traditionally looked at as a sensory system to provide information about detail and spatial awareness, research has also documented that the eyes deliver important photopic information to brain centers which affect hormonal imbalance, diurnal cycle for sleep regulation, and metabolic function. John Ott has described in his research that various wavelengths in the photopic spectrum have significant effects on the growth of plants as well as human biological functions.

The human eye responds to a visual spectrum between 400 nanometers and 700 nanometers. The change in wave length is processed by the brain through the eyes and establishes the perception and interpretation of color. Shorter wavelength is perceived in the blue end of the spectrum while longer wavelength is perceived in the red end of the spectrum. It has also been documented that the retina of the eye is characteristically sensitive to long wavelengths in the central or macular region of the eye, while the peripheral part of the retina is more sensitive to short wavelengths or the blue spectrum.

Research studies document many beneficial color therapy uses. A means of portable color therapy can implement many of these uses in a convenient manner. Successful color therapy use studies include the following applications.

In a non-limiting example, color therapy can be used to enhance reading comprehension. Colored overlay films can optimize reading comprehension. The current invention offers color therapy overlays that are paired or layered with educational, reading or other training software to address learning disabilities and enhance functional reading and comprehension.

Research studies indicate that individually prescribed colored filters have been shown to improve reading performance in people with visual stress. [2]

However, Color overlay selection has largely been a matter of trial and error. [3]

Professor Arnold J. Wilkins has done extensive work with color overlays and has conducted numerous research studies. In one study, Wilkins showed 50% of mainstream students saw improvement and consistently used colored overlays while reading. Wilkins has documented colored overlay benefits for those who suffer from photo sensitive epilepsy, migraines, and other conditions. [4]

Wilkins, et al, teaches that colored overlay diagnostics must match the lighting conditions of the testing procedure in order to attain testing result performance. [5] This, however, reduces the effectiveness of existing testing such as colorimeter testing as it does not match a student's real-world lighting conditions.

Smaller scale color overlay studies have also been performed by other practitioners using various methods. A review of the literature indicates inconsistent results when comparing varying testing standards and use of colored overlays by various manufacturers. Each manufacturer produces overlays of varying transparency levels, producing a range of results in practice. An office or educational setting limited to a certain set of diagnostic overlays may not best meet the needs of the student or user seeking reading enhancement.

The present invention offers a means of computerized testing conditions can be duplicated precisely with the present invention with minor display setting adjustments for devices and screen models, as needed. It offers the ability to tailor a color overlay to the exact color and transparency level best suited to the needs of the student or other user interested in optimizing reading ability.

Some demographics well served by colored overlays include younger readers, the dyslexic, and those with glare sensitivity. Some research-based interventions include colored reading overlays for early readers.

“Coloured reading overlays reduced reading times on the reading test employed here and the size of the reading aid was not crucial to facilitate performance. The largest reductions occurred for the youngest readers, suggesting these aids may be particularly effective for early readers.” [6]

Colored overlays have also been used to enhance reading performance. The studies of regarding Meares Men Syndrome suggest “that the green filter improved reading performance in children with dyslexia because the filter most likely facilitated cortical activity and decreased visual distortions.” [7]

In one study, Wilkins layered multiple color overlay films to best effect. [8] The current invention can rapidly produce accurate digital displays of layered color therapy overlays with little trial and error. It can also document results before a tested student experiences fatigue.

Diagnostic testing to determine the precision color to best alleviate symptoms is often done with bulky and expensive equipment such as Colorimeters. Such equipment is costly for a learning specialist, reading specialist or disability office. The current invention is low cost and computerized.

Some color therapy sequences developed by the claimed invention are established as helpful by research and do not require a custom diagnostic prescription. Some examples of this are sequences for re-establishing functional circadian rhythm and the treatment of Seasonal Affective Disorder. These color therapy sequences may be ready for download as needed. For the diagnosis of customized optimal color therapy sequences, however, the following protocol exists.

To perform the functions described above, an apparatus shown in block diagram in FIG. diagram is utilized. This apparatus utilizes a color display or array 2 which is fed with the display output of a central processing unit 4. This central processing unit 4 may be any computer device such as a desktop computer, laptop, etc., which includes at least a microprocessor, random access memory, a keyboard, a semi-permanent storage system and a color display driver. As an input to the CPU 4 is clinician input 6 which may be the keyboard of the CPU 4 or some other device such as a touch screen, mouse, joy stick, etc. The CPU 4 is capable of providing data output directly to a portable device 8 such as a laptop computer, PDA, etc. Another data output of the CPU 4 goes to a conversion means 10. The conversion means 10 may comprise a program within the CPU 4 for converting data stored in the CPU 4 into a format capable of being handled by other devices such as laptop computer, tablets, smart phones, PDA's etc. belonging to or leased by the patient and then storing it on a CDROM, DVD, tape, cloud storage, etc. In addition, the conversion means 10 may also comprise a means for providing an interface between the CPU 4 and a local area network, internet, phone line, etc.

It should also be apparent to one of ordinary skill in the art that a new and “intelligent” or smart device with more computing capability are created such as intelligent VCRs, CDROM players and DVD players that the function of the device described above and the patients device could incorporate or in fact be such “intelligent” devices.

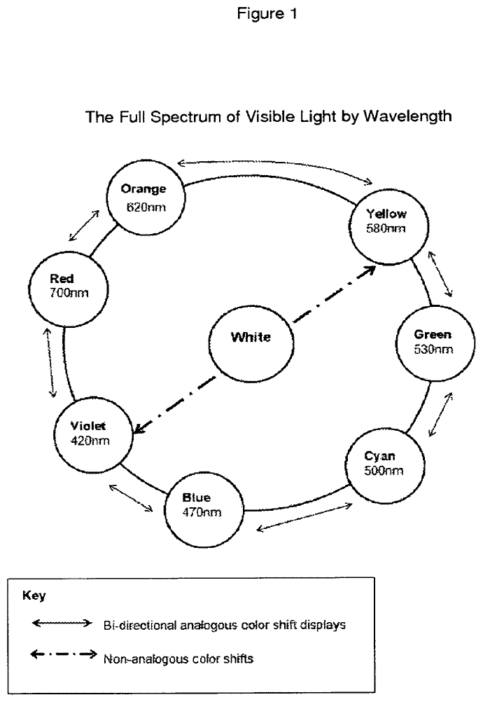

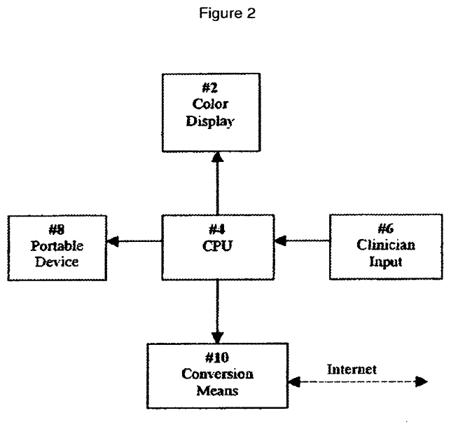

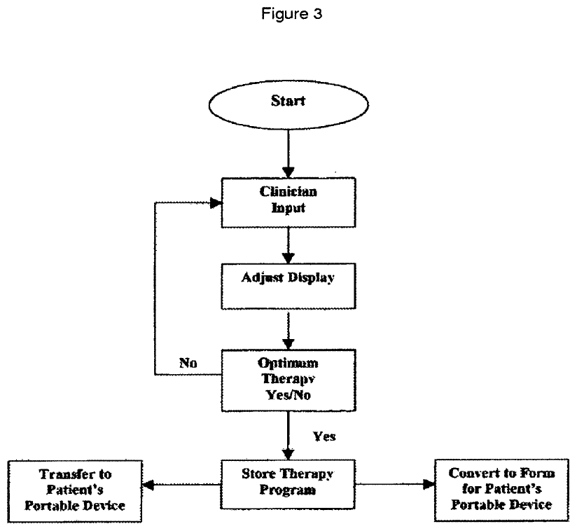

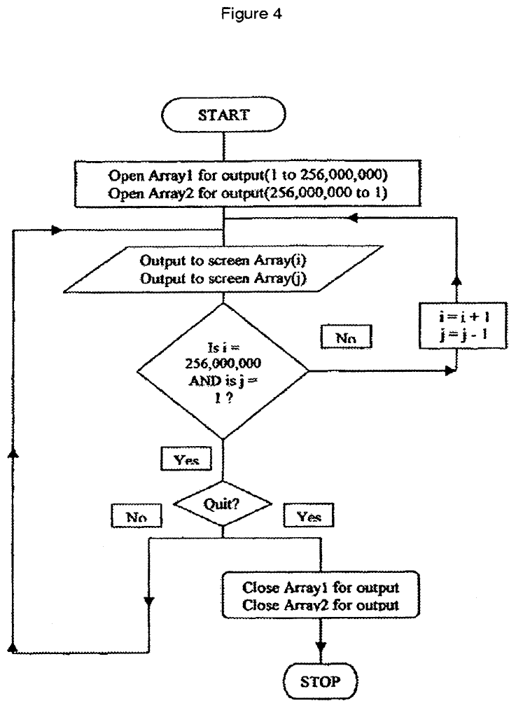

Referring to FIGS. 1-4, a mode of therapy for persons who have experienced a neurological event, neurologically-based learning disability or other learning disability impacting reading or other neurological dysfunction will be described in the numbered paragraphs 1-6 below.
1. The patient, student or other user will be seated before a visual display 2 such as a television, CRT or LCD monitor, or portable tablet, smart phone, or Virtual Reality gear. These will provide specific wavelengths that are perceived by the visual process as variation in color. The patient, student or end-user will be seated between 15-25 inches from the display, monitor or screen.
2. The display 2 will be adjusted using the clinician input 6 to provide initially a balance between the blue or short wavelength end of the visual spectrum and the red or long wavelength of the visual spectrum. For patients who have experienced a neurological event or cause that interferes with the ambient visual process, treatment will then be shifted to the blue end of the spectrum by the clinician. The patient will be given three five-minute therapy sessions exposed to short nanometer wavelengths of light.
3. The apparatus will then be adjusted through the clinician input 6 to the CPU 4 to shift from the blue end of the spectrum toward longer wavelengths. The design of the apparatus will enable the clinician to develop gradation shifting across the spectrum in a variety of ways as shown in FIG. 1 such that blue can be shifted in wavelength toward various spectrum portions such as green, yellow, or red in accordance with the flow diagram of FIG. 4. This will enable the clinician to be very specific in delivering the direction of the therapy toward specific aspects of motor function, cognitive function, or higher perceptual processes. For example, shifting from blue to red will be oriented to bring spatial relationships to development of figure/ground relationships and perceptual constancy. Shifting to the yellow end of the spectrum will have more specific function related to movement, object localization, and perceptual transformations. Shifting from blue toward the green end of the spectrum will be more related to affecting those patients who are experiencing a highly focalized nature to their vision such as in autism where the visual system will fragment the world into detail or parts. While FIG. 1 is described in terms of at least 256,000,000 possible colors, it should be apparent to one of ordinary skill in the art that the present invention would function with less color combinations.
4. For those patients who are experiencing neurological dysfunction as related to learning disabilities and attention deficit disorders, the color or wavelength variation will be shifted from red, yellow, or green toward the green-blue to blue end of the spectrum in a similar manner described in method one (1). The temporal component will also be altered related to the critical fusion frequency of the focal or ambient process.
5. If a reading ability test to assess possible optimal color overlays is to be performed, an additional step is added to method four (4). While the student, patient or end-user is seated before the shifting color therapy, a grade-suitable textual overlay will be displayed. The combined textual and color therapy display will be shown to the student, patient or end-user. Reading speeds and accuracy may be assessed manually or documented via the program.
6. The wavefront modulation system is designed as a therapeutic mechanism to treat these visual problems that up to this time no methodology or instrument invention has been found to improve function. Referring further to FIG. 3, in operation the clinician will start the apparatus and provide initial operator input into the apparatus. Based on this initial input by the educator, learning specialist or clinician, the color display 2 will be adjusted to provide the correct colors, shift and animation effect. After the patient, student or end-user has been exposed to the color display 2 for the required time, typically 5 minute sessions, the effect of the color therapy will be observed by the clinician who will determine whether or not the therapy is now at an optimum level. If no, there will be further adjustments made and if yes, the optimum color therapy program which has been developed during the session or sessions will be stored in the CPU 4. So that the patient, student or end user can utilize this optimum color therapy program developed during the session or sessions, the optimum color therapy program for this particular patient which is stored in the CPU 4 will be then either directly transferred to the portable device 8 of the patient or sent to the conversion means 10 for conversion either to a program in the format usable by the portable device of the patient or into a format which can be transferred over the internet, a local area network, downloaded from website, mobile application store or cloud, to be accessed by a patient, student or end user at a remote location. The control program for the operation shown in FIG. 3 can be easily created by one of ordinary skill in the art based upon the flow diagram of FIG. 3. By providing the patient, student or user with the means for utilizing the optimum color therapy program developed specifically for them on a device in the possession of the patient, student or user and at a location and times of the user's choice, many of the disadvantages of the prior art can be overcome.
7. Specific color therapy sequences have been documented as helpful for various health, wellness, lifestyle and educational issues. These proven sequences do not require a customized prescription to enact benefit. Such programs can be available for download as seen fit, including by recommendation of practitioners learning specialists, and other educational professionals, as these have already demonstrated usefulness for educational, health, wellness, and lifestyle use. Some examples include the aforementioned colored overlays to enhance functional reading, blue light negating overlay color sequence of a specific wavelength of ˜590-625 nm running concurrently with other programs and applications, green light nm sequences to optimize reading speed and reduce glare sensitivity, and color shift sequences for Seasonal Affective Disorder, sleep disturbance, circadian rhythm issues due to jet lag and Shift Work Sleep Disorder (SWSD), and other similar self-limiting wellness issues.
   Still further, the apparatus and method of the present invention provides one or more of the following:
   1. A method of diagnosis of optimal color wavelengths for devising an exact therapeutic or beneficial range including nanometer specifications and hue-saturation to prescribe for individuals with a wide range of visual problems, including but not limited to learning disabilities and neurological problems.
   2. A computer-implemented method for assisting a user in cognitive and vision rehabilitation, as well as the rehabilitation and assistance of learning disabilities via a handheld device to assist the visually impaired, learning-impaired, as well as those in need of cognitive rehabilitation.
   3. Adaptations of current rehabilitation software as outlined in other patents by the same inventors (Dara C. Medes, Heather Lyn Medes) to be downloaded onto a handheld smart tablet or device.
   4. A website to make said software suite available for download onto a handheld device.
   5. A CD, DVD and any such other recordable medium devices to hold such software for distribution. Other storage mediums suitable for program transfer include cloud storage, website download, and casting.
   6. The software processes of converting rehabilitation software from dumb-terminal non-portable systems to software that is portable via any handheld or telecommunication device.
   7. Treatment methods in or outside of traditional rehabilitation setting by using said software suite to do rehabilitation in a setting and schedule most convenient to the patient.
   8. Treatments traditionally associated with light therapy including but not limited to headaches, asthma, depression, weight problems, adrenal and hormonal imbalances, dermatological enhancement, cosmetic enhancement, vision therapy, reduction of edema, improved blood flow, bilirubin regulation, amongst others, can be available in a portable form that may not require constant office supervision or costly equipment.
   9. Treatment services traditionally restricted to directly out of a doctor's office may now be monitored via wired or wireless telecommunication or processing devices, including any other form of transmission technology, or in a doctor's or therapist's office setting. This allows for treating the house bound or those geographically far away in or out of a traditional office treatment setting.
   10. Treatments that may or may not require a doctor's supervision but are often recommended by a doctor, educator or other practitioner include reading enhancement for learning disability, circadian rhythm sleep disturbance such as Night Worker Sleep Disorder and jet lag, Seasonal Affective Disorder, color therapy sequence aids for RMT/breathing rehabilitation, colored overlays to block device projected blue light and other wellness, health, relaxation, entertainment or enjoyment uses for color therapy.
   11. Color therapy programs that are documented as useful for a specific self-limiting problem can be available to end users without a diagnostic review. These include color therapy sequences as a means for enjoyment, relaxation, and entertainment, as well as Respiratory Muscle Retraining enhancement guides, pain management, cognitive training, ocular precision sports training software restorative sleep function programs as outlined above.

The above description is for the purpose of teaching the person of ordinary skill how to practice the present invention, and it is not intended to detail all obvious modifications and variations of it, which will become apparent to the skilled worker upon reading the description. It is intended, however, that all such obvious modifications and variations be included within the scope of the present invention, which is defined by the following claims.

The invention claimed is:

1. A color shift therapy apparatus comprising: a control means; a color display means for being viewed by a patient; and a driving means for at least providing color shifting signals to said color display in response to said control means for displaying gradation shifting colors on said color display means; whereby in response to said control means a color shift from one preselected color to another preselected color is repeatedly performed while changing said one pre-selected color and said another preselected color until an optimum color therapy program of the correct colors and gradation shift is determined; whereby said gradation shift between the correct colors is to be repeatedly displayed in therapy.

2. The color shift therapy apparatus according to claim 1 wherein said control means comprises a clinician input CaS.

3. The color shift therapy apparatus according to claim 2 wherein said clinician input means comprises at least one selected from the group consisting of a keyboard, a mouse, a joystick and a touch screen.

4. The color shift therapy apparatus according to claim 1 wherein said means for providing color shifting signals comprise a central processing unit.

5. The color shift therapy apparatus according to claim 4 wherein said color display means is selected from the group comprising of CRT, LCD, Virtual reality headset or comparable gear, and a television.

6. The color shift therapy apparatus according to claim 5 wherein said display means is capable of displaying 256 million possible colors.

7. The method for providing color shift therapy with the apparatus of claim 1, said method comprising the steps of placing a patient in front of the color display;
providing initial clinician input to the control means to display colors and gradation color shifts on the color display;
having said patient, student or other end-user view said colors and gradation color shifts on said color display;
determining if said colors and gradation color shifts have a desired effect on said patient, student or other end-user;
and changing said colors and gradation color shifts until said desired effect occurs;
whereby said gradation color shifts between said colors, having said desired effect, is to be repeatedly displayed in therapy.

8. The method according to claim 7 further comprising creating and storing an optimum therapy program for said patient, student or user when it is determined that said colors, color overlay or overlays and color shifts are having the desired effect on patient.

9. The method according to claim 8 further comprising providing one optimum therapy program to said patient, student or user for use on said user's own remotely usable apparatus.

10. The method according to claim 7 further comprising the steps of transmitting said optimum color therapy program to a remote device.

11. The method according to claim 7 further comprising the step of providing another concurrent program display combined with the optimum color therapy program displayed as a partially transparent overlay on said concurrent program. Said concurrent program includes other effective treatment programs, the display of educational, wellness, rehabilitative, productivity or lifestyle programs, or the display of texts or data, image or animation, film, 3D animation and renderings, film effects or motion displays.

12. The color shift therapy apparatus according to claim 1 in conjunction with the method according to claim 11, whereupon also combining the claim 7 comprised of optimized color therapy partially transparent programs overlaying other applications or programs. These applications include effective educational or treatment programs, text content and data, image or animation, 3D animation and renderings, film, film effects or motion displays, where said display is combined with an audio component and/or audio response feedback.

13. The color shift therapy apparatus according to claim 1 in conjunction with the method according to claim 11, whereupon also combining the claim 7 comprised of optimized color therapy partially transparent programs overlaying other applications or programs where Virtual Reality headgear is used to display multi-dimensional color therapy projections with or without other useful applications.

14. The color shift therapy apparatus according to claim 1 further comprising a means for transmitting said optimum color therapy program to a remote device.

15. The color shift therapy apparatus according to claim 13 wherein said optimum color therapy program is transmitted to a remote device via Internet.

16. The color shift therapy apparatus according to claim 1 further comprising a means for storing said optimum color therapy program and for transferring said optimum color therapy program to a detachable apparatus for using said optimum color therapy program at remote locations by said patient.
